(19) United States
(12) Patent Application Publication
(10) Pub. No.: US 2007/0255198 A1
(43) Pub. Date: Nov. 1, 2007

(54) Substance Delivery Device

(75) Inventors: Weng Yee Leong, Malvern (GB); Leigh Trevor Canham, Malvern (GB)

Correspondence Address: NIXON & VANDERHYE, PC, 901 NORTH GLEBE ROAD, 11TH FLOOR, ARLINGTON, VA, 22203, US

(73) Assignee: PSIMEDICA LIMITED, Worcestershire (GB)

(21) Appl. No.: 11/663,056

(22) PCT Filed: Sep. 12, 2005
(86) PCT No.: PCT/GB05/03523

§ 371 Date: Jun. 15, 2007

(30) Foreign Application Priority Data
Sep. 17, 2004 (GB) 0420676.9

(51) Int. Cl. A61M 5/14

(52) U.S. Cl. 604/65

(57) ABSTRACT

The present invention relates to a substance delivery device comprising: (a) an electronic component, (b) a carrier component, and (c) a beneficial substance, the electronic component comprising: (i) a timer means for generating a carrier activation electrical signal that is time dependent; (ii) an electronic carrier activation means for releasing the beneficial substance in response to the carrier activation electrical signal; (iii) a power supply; and (iv) a substance delivery device activation means, for activating the device, having a construction such that once the substance delivery device is activated the electronic carrier activation means releases the beneficial substance in response to the carrier activation electrical signal. The substance delivery device is of particular value in the treatment of circadian, infradian, or ultradian cycle conditions.

The present invention relates to a new substance delivery device comprising a timing device and a silicon carrier component for delivering a therapeutic substance. The invention further relates to a new substance delivery device, comprising silicon carrier component, for treating medical conditions that have a circadian, infradian, or ultradian cycle.

All human beings have an in-built ability to approximately determine the time of day, the part of the brain that is responsible for this function being the suprachiasmatic nucleus. Subsequent research has shown how this nucleus is controlled, and how it sends information about time to the rest of the body. It is the objective of chronotherapy to coordinate medical and vetinary treatment with biological rythms to maximise benefit and/or to minimise side-effects and risk. There are three types of biological rhythm: (a) circadian (having a 24 hour cycle), infradian (having a cycle of greater duration than 24 hours), and ultradian (having a cycle shorter than 24 hours).

Two diverse examples of circadian conditions are jet lag, and withdrawal symptoms resulting from drug addiction. Diverse examples of infradian conditions include fertility and seasonal affective disorder. Medical conditions that are subject to a circadian cycle include hypertension, rheumatoid arthritis, diabetes, asthma, Parkinson's disease, chronic ulcers, GERD, and cancer. In humans post surgical death is most likely at 1 am, blood pressure reaches a minimum at 3 am, and asthma is at its worst at 4 am. Chronotherapy takes account of a person's biological rythms in determining the timing of medication. For example, the chronotherapeutic delivery of a bisoprolol formulation, has been described as effective in minimising the risk of acute cardiovascular episodes (U.S. Pat. No. 6,733,789).

Some types of medical treatment require the repeated administration of a drug over an extended interval. This can result in problems as a result of the patient forgetting, or being unable to comply with this requirement. However, even if the drug is to be taken repeatedly over a relatively short time it is still possible for a patient to forget to take the drug at a particular time.

A number of prior art devices have been devised in an attempt to deal with time dependent conditions. Such devices may be implanted, or administered via the gastrointestinal system.

The beneficial substance to be administered may be located in one or more reservoirs, a barrier of erodable material, such as a biodegradable polymer, being located between the beneficial substance and the exterior of the device. Once implanted, or otherwise administered, the barrier erodes until the active material is released after an interval determined by the erosion rate of the polymer and the dimensions of the barrier.

In addition to the use of erodable materials, control of release can be achieved by using certain carrier materials including porous carrier materials; the rate of release being determined by diffusion through the material of the carrier or by diffusion through the pore system.

If the administration is via the alimentary canal, it is often necessary to release the drug at a particular location. If this is the case then the barrier material can be selected so that it only erodes at this location; for example it can selected so that it only erodes in the gastric environment, or that it only erodes in the intestinal environment.

Pharmaceutical products comprising sensors, radiotransmitters, and radioreceivers, have also been described, such devices allowing the monitoring of the environment, or location in the gastrointestinal system, so that the beneficial substance can be released at a particular location with much greater accuracy.

Not all prior art products require the erosion of a carrier material, or diffusion of the drug. Some have been described in which a mechanical actuator is used to open a reservoir in which the drug is located. The signal to open the reservoir being sent to the actuator via a radio receiver.

The following documents provide relevant background information: U.S. Pat. No. 4,564,363; U.S. Pat. No. 6,632,216; U.S. Pat. No. 5,279,607; WO 98/00107; U.S. Pat. No. 6,123,861; U.S. 20030190360; WO 01/028529; EP 0239 605 B1; WO 04/066903; U.S. 2004/0158194 A1; and U.S. Pat. No. 4,601,707. U.S. Pat. No. 4,564,363 describes a delayed release ruminant dosage form; U.S. Pat. No. 6,632,216 describes an ingestible device for delivering a substance to an identifiable location; U.S. Pat. No. 5,279,607 describes an ingestible capsule for delivery of a medicament to the alimentary canal; WO 98/00107 describes a microchip device comprising a plurality of reservoirs that controllably release a drug; U.S. Pat. No. 6,123,861 describes a method of fabricating a microchip device for the release of a substance from a number of reservoirs; U.S. 20030190360 describes a delayed release solid dosage form, WO 0128529 describes orally administrable pharmaceutical products comprising porous silicon; EP 0239 605 B1 describes methods of fabricating a structure for the controlled release of a substance; U.S. 2004/0158194 describes a device for controlled release, comprising a reservoir containing a drug and an electronic drug release mechanism; U.S. Pat. No. 4,601,707 describes a device for injection of a medicament subcutaneously; WO 04/066903 describes an apparatus for drug administration comprising an ingestible capsule having an environmentally sensitive mechanism.

There are a variety of problems with the gastrointestinal devices described above. Many devices based on erosion of a barrier, prior to release of a substance may lack the required accuracy of time and location of release. This is because the gastrointestinal environment and transit time can vary from subject to subject or vary with the diet of the subject. Devices that comprise electronic circuitry offer the possibility of improving this accuracy, but are often very expensive, bulky, and/or complex, because of the inclusion of sensors, radio transmitters, and radio receivers.

An objective of the present invention is to solve at least some of the above mentioned problems. A further objective of the present invention is to provide a device that can deliver a beneficial substance at a specific time, times, or range of times, independent of when the substance delivery device is administered. A yet further objective of the present invention is to provide a substance delivery device that can deliver a beneficial substance at a specific time, times, or range of times, and that is inexpensive, small, and that has a relatively simple construction.

According to a first aspect, the present invention provides a substance delivery device comprising: (a) an electronic timer means for generating a time dependent electronic signal, (b) a substance delivery device activation means for activating the substance delivery device, (c) a beneficial substance, and (d) a beneficial substance release means .for releasing the beneficial substance in response to the time dependent electronic signal, once the substance delivery device has been activated.

The substance delivery device may comprise an electrical power supply to supply electrical power to the timer means, the beneficial substance release means, and/or the substance delivery device activation means.

The substance delivery device activation means may comprise a means for activating the substance delivery device when the substance delivery device is located in the mouth of a person or animal. The substance delivery device activation means may comprise a means for activating the substance delivery device when the substance delivery device is located in the gastric environment of a person or animal. The device activation means may comprise a means for activating the substance delivery device when the substance delivery device is located in the intestinal environment of a person or animal. The device activation means may comprise a means for activating the substance delivery device when the substance delivery device has been implanted in a person or animal.

Before being administered to a patient, the substance delivery device may be located in a package. The device activation means may comprise a means for activating the substance delivery device when the substance delivery device has been removed from the package. The device activation means may comprise a means for activating the substance delivery device when the package has been opened.

If the substance delivery device is fabricated at a factory, and is for use in a particular time zone, then the timer means may be programmed at the factory to generate an electronic signal at a particular time of day in that time zone. The removal of the substance delivery device from its packaging, or the saliva in the mouth of the patient may cause the device activation means to activate the substance delivery device. Once the substance delivery device has been activated the beneficical substance release means is able to release the beneficial substance in response to the time dependent electronic signal. The time between the activation of the substance delivery device and the release of the beneficial substance may vary depending upon when the patient consumes the substance delivery device. The time of beneficial substance release is, however, independent of the time of consumption.

For example, the timer means may generate an electronic signal at 3 am every day after its fabrication. However, until the substance delivery device is activated, release of the beneficial substance does not occur. The beneficial substance will only be released once the substance delivery device has been consumed, and then release will occur at 3 am irrespective of when the substance delivery device is administered. This means that provided the device is administered within a relatively broad range of times it can be activated when the device is likely to be located in, say, the intestines of a patient.

This differs from many prior art orally administrable devices, that depend upon the transit time and gastrointestinal conditions of the subject to determine the time of release of a drug. The substance delivery devices of the present invention can be made to deliver a drug at a particular time, irrespective of conditions, that can vary greatly from person to person. The invention is therefore of great value in the treatment of conditions that require a substance to be reliably delivered to a subject at a particular time, or particular times.

The substance delivery device may comprise a carrier component. The carrier component may comprise a carrier material. The beneficial substance release means may comprise the carrier component.

The carrier material may be at least partly porous, the beneficial substance being located in at least some of the pores of the porous carrier material. The carrier material may be at least partly polycrystalline, the beneficial substance being located in at least some of the space between the crystallites. The beneficial substance may be distributed through at least some of the carrier material.

The substance delivery device may comprise a reservoir, the beneficial substance being located in the reservoir. The substance delivery device may comprise a barrier that, when in use, is located between the reservoir and the surroundings of the substance delivery device. The barrier may comprise the carrier material. The substance delivery device may comprise two or more reservoirs, the beneficial substance being located in one or more of the reservoirs.

The carrier material may comprise silicon. The carrier material may comprise porous and/or polycrystalline silicon. The carrier material may comprise one or more of: microporous silicon, mesoporous silicon, and macroporous silicon. Microporous silicon contains pores having a diameter less than 20 Å; mesoporous silicon contains pores having a diameter in the range 20 Å to 500 Å; and macroporous silicon contains pores having a diameter greater than 500 Å.

The carrier material may comprise porous silicon having a porosity between 2% and 95%. The carrier material may comprise porous silicon having a porosity between 4% and 90%. The carrier material may comprise porous silicon having a porosity between 4% and 70%. The carrier material may comprise porous silicon having a porosity between 10% and 70%.

The structure and composition of the carrier material may be such that it erodes when it is implanted in a human or animal. The structure and composition of the carrier material may be such that it erodes when it is in the gastric environment of an animal or human. The structure and composition of the carrier material may be such that it erodes when it is in the intestines of an animal or human. The structure and composition of the carrier material may be such that it is substantially resistant to the gastrointestinal tract of an animal or human.

The beneficial substance release means may comprise a means for heating the carrier material.

The carrier material may comprise porous and/or polycrystalline silicon. As described in WO 9706101 and WO 0128529, which are herein incorporated by reference in their entirety, porous and polycrystalline silicon has been found to be resorbable in biological environments. The rate of erosion of the porous or polycrystalline silicon may be increased by electrically heating the silicon. If a beneficial substance is located in the pores of the porous silicon, or if the silicon is used as a barrier to separate the beneficial substance from the surroundings of the substance delivery device, then such heating may cause the beneficial substance to be released.

The carrier material may comprise porous and/or polycrystalline silicon and the beneficial substance release means may comprise a means for increasing the pH in the region of the porous and/or polycrystalline silicon carrier material. The carrier material may comprise mesoporous silicon and the beneficial substance release means may comprise a means for increasing the pH in the region of the mesoporous silicon carrier material.

The means for increasing the pH in the region of the porous and/or polycrystalline carrier material may comprise first pH electrode and a second pH electrode. The means for increasing the pH may have a construction such that the first pH electrode is closer to the carrier material than the second pH electrode. The means for increasing the pH may have a construction such that the first pH electrode is closer to at least part of the carrier material than the second pH electrode. The means for increasing the pH may have a construction such that any point on the surface of the first pH electrode is closer to any point within the carrier material than any point on the surface of the second pH electrode.

The substance delivery device may have a construction such that neither of the first and second pH electrodes is in direct physical contact with the porous silicon. The substance delivery device may have a construction such that no point in or on either of the first and second pH electrodes is less than 10 mm from any point in or on the porous silicon. The substance delivery device may have a construction such that no point in or on either of the first or second pH electrodes is less than 1 mm from any point in or on the porous silicon. The substance delivery device may have a construction such that no point in or on either of the first and second pH electrodes is less than 100 microns from any point in or on the porous silicon.

When in use the potential on the first pH electrode may be negative relative to that on the second pH electrode. The substance delivery device may have a construction such that, implanted or consumed, at least part of both the first and second pH electrodes are in contact with a physiological electrolyte. The substance delivery device may have a construction such that, when in use, at least part of the surface of the carrier material is in contact with a physiological electrolyte.

The application of a negative bias to the first pH electrode may cause the pH at the surface of the carrier material to increase in the region of the carrier material. This may occur as a result of the migration of ions, such as H⁺ and OH⁻ ions, within the physiological electrolyte.

The substance delivery device may have a construction such that no potential difference is applied between the first pH electrode and the carrier material.

The means for increasing the pH may comprise a third pH electrode, and a fourth pH electrode, the fourth pH electrode being in direct physical and electrical contact with at least part of the porous silicon carrier material. The substance delivery device may have a construction such that, when in use, the fourth pH electrode is negatively biased relative to the third pH electrode. The substance delivery device may have a construction such that, when immersed in a physiological electrolyte, at least part of the third pH electrode and at least part of the porous silicon carrier material are in contact with the electrolyte. The silicon carrier material may comprise a low porosity region, and a high porosity region, the beneficial substance being located in at least some of the pores of the low porosity region. The low and high porosity regions may be arranged such that dissolution, by the physiological electrolyte, of the high porosity region occurs more rapidly than the low porosity region when a potential difference is applied to the third and fourth pH electrodes. A beneficial substance may be disposed in at least some of the pores of the low porosity region in such a way that dissolution of the high porosity region results in release of the beneficial substance. The low porosity porous silicon and the high porosity porous silicon may be arranged such that at least some of the pores, of the low porosity porous silicon, in which the beneficial substance is located, are blocked by the high porosity porous silicon.

The rate of corrosion of porous and polycrystalline silicon can be increased by increasing the pH of the biological environment in which it is located. If the beneficial substance is located in the pores of the porous silicon, or if the silicon is used as a barrier to separate the beneficial substance from the surroundings of the substance delivery device, then such an increase in pH may cause the beneficial substance to be released by increasing the rate of erosion.

The beneficial substance release means may comprise a means for applying a positive or negative potential, relative to earth potential, to the carrier material. The beneficial substance release means may comprise a means for applying a positive potential, relative to earth potential, to the carrier material.

The erosion of porous and polycrystalline silicon in biological environments is accelerated by the application of a positive bias to the silicon. If a beneficial substance is located in the pores or the porous silicon or if the silicon is used as a barrier to separate the beneficial substance from the surroundings of the substance delivery device, then the application of such a bias may cause the beneficial substance to be released.

If the carrier material comprises porous silicon, and a beneficial substance, located in at least some of the pores of the porous silicon, is polar or ionic, then the application of an electrical potential to the porous silicon may cause the beneficial substance to exit the porous silicon as a result of electrostatic repulsion between the porous silicon and the beneficial substance. The semiconducting properties of the porous silicon facilitate this form of beneficial substance release.

The beneficial substance release means may comprise a means for generating a gas in the region of the carrier material. The carrier material may comprise a porous material and the beneficial substance release means may comprise a means for generating a gas in the pores of the porous material.

If the beneficial substance is located in at least some of the pores of the porous carrier material, then the action of the gas pressure on the beneficial substance may cause the beneficial substance to exit the pores. Alternatively the generation of the gas may cause the porous carrier material to fracture, thereby releasing the beneficial substance.

The beneficial substance may comprise a multiplicity of beneficial substance molecules. At least some of the beneficial substance molecules may be bound to the surface of the carrier material. At least some of the beneficial substance molecules may be chemically bound to the surface of the carrier material.

The beneficial substance release means may comprise a means for releasing any beneficial substance molecules that are bound to the surface of the carrier material.

The timer means may be programmed such that and/or have a construction such that, when in use, the electrical signal is generated at a particular time of day.

The timer means may be programmed such that and/or have a construction such that, when in use, the electrical signal is generated is periodic. The period of the electrical signal may be selected from one or more of: one minute, one hour, one day, one week, one month, and one year. The period of the electrical signal may be between one minute and one year. The period of the electrical signal may be between one minute and one day. The period of the electrical signal may be between one day and one week.

The electrical signal may comprise an electrical current between two parts of the substance delivery device. The electrical signal may comprise a potential difference between two parts of the substance delivery device.

The substance delivery device activation means may comprise an activation electrical switch having an on state and an off state, the substance delivery device being activated when the activation electrical switch has been switched to an on state. The substance delivery device may have a construction such that when the activation electrical switch has been switched to an on state, current may flow to, and/or a potential difference may be applied to, the beneficial substance release means as a result of the generation of the electrical signal by the timer means.

The activation electrical switch may comprise two or more switch electrodes.

The substance delivery device may have a construction such that activation electrical switch is switched to an on state by one or more of: (i) contact with saliva of a person or animal; (ii) contact with the gastric environment of a person or animal; (iii) contact with the intestinal environment of a person or animal; (iii) implantation of the substance delivery device in a person or animal; (iv) removal of the substance delivery device from a package.

If the substance delivery device comprises an excipient coating, then contact between the device activating electrical switch and the gastrointestinal environment, and/or saliva, may be preceded by dissolution, and/or distortion, of the excipient coating.

The activation electrical switch may have a construction such that it is switched to an on state by contact with a physiological electrolyte. The activation electrical switch may have a construction such that it is switched to an on state by the mechanical action of removing the substance delivery device from the package. The activation electrical switch may have a construction such that it is switched to an on state when substance delivery device reaches a predetermined temperature. The predetermined temperature may be the normal body temperature of the animal or human subject. The activation electrical switch may comprise a thermocouple.

For the absence of doubt a physiological electrolyte is an aqueous electrolyte that is produced by, or involved with, a physiological process of an animal or human. Contact between the switch electrodes of the activation electrical switch may, when the substance delivery device is in use, result in an electrical current passing through the physiological electrolyte, between the two or at least two of the switch electrodes of the activation electrical switch.

The timer means may be programmed such that and/or have a construction such that, when in use, the electrical signal is generated at two or more times of day.

The timer means may be programmed such that and/or have a construction such that, when in use, the electrical signal is generated during an interval of time.

The timer means may be programmed such that and/or have a construction such that, when in use, the electrical signal is generated during two or more intervals of time.

The timer means may be programmed such that and/or have a construction such that, when in use, the electrical signal is generated at a number of times during a week.

The timer means may be programmed such that and/or have a construction such that, when in use, the electrical signal is generated at a particular range of times during a week.

The timer means may be programmed such that and/or have a construction such that, when in use, the electrical signal is generated at a number of times during a month.

The timer means may be programmed such that and/or have a construction such that, when in use, the electrical signal is generated at a particular range of times during a month.

The timer means may be programmed and/or have a construction such that it generates the electrical signal that is dependent upon the time of day for a particular time zone.

The substance delivery device may comprise an electronic component.

The electronic component may substantially consist of: (i) a timer means for generating a time dependent electrical signal; (ii) a beneficial substance release means for releasing the beneficial substance in response to the electrical signal received from the timer means; (iii) an electrical power supply; and (iv) electrical connections between the timer means, beneficial substance delivery means, and power supply.

The electronic component may substantially consist of: (i) a timer means to provide an time dependent electrical signal, (ii) beneficial substance release means for releasing the beneficial substance in response to an electrical signal received from the timer means; (iii) a device activation means, (iv) an electrical power supply; and (v) electrical connections between the timer means, beneficial substance release means, device activation means, and power supply.

The substance delivery device may substantially consist of the electronic component, carrier material, and beneficial substance. The substance delivery device may substantially consist of the electronic component, carrier material, beneficial substance, and an excipient coating.

The excipient coating may comprise any substance that is commonly used to coat pharmaceutical products. Such pharmaceutical coatings may be used to make the product more palatable to the subject. The excipient coating may comprise one or more of: sucrose, gelatine, lactose, dextrose, starch, mannitol, cellulose, cellulose derivatives, dicalcium phosphate, tricalcium phosphate, calcium carbonate, and calcium sulphate.

The timer means may comprise a microcontroller chip, a clock chip, and an oscillator crystal.

The timer means may comprise one or more of the following IC chips: RX-4045SA (Epson RTC chip), DS2417 (Dallas RTC chip), PCF8563 (Philips RTC chip), and PIC16F877A (Microchip PIC microcontroller).

The timer means may comprise one or more of: a clock chip, a crystal oscillator, a CMOS ring oscillator, and a polycrystalline silicon resonator.

The timer means may comprise an Epson 32.768 kHz quartz oscillator.

The timer means may comprise a quartz crystal oscillator and a frequency divider.

The timer means may comprise a microcontroller. The microcontroller may be a 4-bit, 8-bit, or 16-bit microcontroller. The timer may comprise an SGS Thomson Model NE555N or a TS555/6CN Low power timer IC.

The timer may comprise a microcontroller in bare die form.

The power supply may comprise a battery. The power supply may comprise a button battery and/or a thin film microbattery.

The power supply may comprise one or more of: a mercury oxide battery, a silver oxide battery, an alkaline/manganese dioxide battery, a zinc air battery, a lithium battery, a carbon-zinc battery, and a zinc chloride battery.

For the purposes of this specification the term lithium battery includes Li/FeS₂ and Li/MnO₂ batteries.

Because the operation of the electronic component, according to the present invention, depends only upon the presence of a timer means, beneficial substance release means, a device activation means, and a power supply, it is possible for its dimensions to be much smaller than prior art devices. This simplicity also allows the cost of the device to be markedly lower than that of prior art devices.

The substance delivery device may comprise a silicon chip, the electronic component being formed on at least part of the surface of said chip.

The total volume of the substance delivery device may be between 1 and 100 mm³. The total volume of the substance delivery device may be between 1 and 50 mm³. The total volume of the substance delivery device may be between 10 and 50 mm³.

The total volume of the electronic component may be between 1 and 90 mm³. The total volume of the electronic component may between 1 and 40 mm³. The total volume of the electronic component may be between 10 and 40 mm³. The total volume of the electronic component may be less than 40 mm³.

The substance delivery device may comprise a silicon chip, and the carrier material may comprise porous silicon, the porous silicon being integral with the silicon chip.

The porous silicon carrier and at least part of the electronic component may be formed from a single sample of silicon. The porous silicon carrier and at least part of the electronic component may be formed in a single sample of bulk crystalline silicon.

The porous silicon may be fabricated from the same material from which the electronic component is at least partly formed, allowing the size of the substance delivery device to be kept to a minimum.

For the purposes of this specification a “beneficial substance” is something beneficial overall: it could be a toxin toxic to undesirable cells/to interfere with an undesirable physiological process. For example, anti-cancer substances would be considered “beneficial”, even though their aim is to kill cancer cells. The beneficial substance may be selected from one or more of: an alpha or beta adrenergic blocking agent, a bronchodilator, a histamine H2 antagonist, a non steroidal anti inflammatory agent, a corticosteroid, a beta2 adrenoreceptor agonist, an ACE inhibitor, a calcium channel blocker, an analgesic, an immunosuppressant, an antibiotic, and an antiproliferative agent.

The beneficial substance may be selected from one or more of: fluoxetine, sertraline, bupropion, busalfan, ciclosporin, ciprofloxacin, dexamethasone, diazepam, digoxin, dipyridamole, indometacin, isorbide dinitrate, ketoprofen, lithium, lorazepam, nifedipine, nortriptyline, rifampicin, sodium salicylate, sulfamethoxazole, theophylline aminophylline, triazolam and valproic acid.

More preferably, the beneficial substance may be selected from one or more of: paroxetine, venflaxine, bisoprolol, propranolol, metoprolol, verapamil, diltiazem, theophylline, salmeterol, nizatidine, prednisolone, cyclosporine A, salbutamol, ibuprofen, lignocalne, amitriptyline, ketoprofen, diclofenac, isosorbide mononitrate, levodopa, insulin, carboplatin, cisplatin, mitoxantrone, fluorouracil, doxorubicin, chlorambucil, and melatonin.

The beneficial substance may comprise lumiracoxib and/or a cyclooxygenase-2 (COX-2) selective inhibitor.

There are a number of beneficial substances that are effectively absorbed at a range of locations in the intestines. Substance delivery devices according to the present invention are particularly useful for the delivery of such beneficial substances at a particular time, times, or range of times. If the timer means is programmed to generate a carrier activation electrical signal at 3 am, for example, then provided the substance delivery device is at a place in the intestines where the beneficial substance may be absorbed, treatment will be effective. The new substance delivery device is therefore particularly of value, relative to prior art devices, since treatment is much less dependent on the time of consumption or the gut transit time.

According to a further aspect the invention provides a medical kit comprising a package and one or more substance delivery devices as defined in any of the above mentioned aspects.

The device activation means may comprise a means for detecting when the or at least one of the substance delivery devices has been removed from the package.

According to a further aspect the invention provides a substance delivery device as defined in any of the above mentioned aspects for use in one or more of the following medical conditions: asthma, rheumatoid arthritis, hypertension, obesity, type II diabetes, cancer, ulcers, Parkinson's disease, GERD, and allergies.

According to a further aspect the invention provides a substance delivery device as defined in any of the above mentioned aspects for use in one or more of the following conditions: fertility, seasonal affective disorder, jet lag, drug abuse, and disrupted sleep patterns.

According to a further aspect the invention provides use of a substance delivery device as defined in any of the above mentioned aspects for the manufacture of a medicament for the treatment of one or more of the following medical conditions: asthma, rheumatoid arthritis, hypertension, obesity, type II diabetes, cancer, ulcers, Parkinson's disease, and allergies.

According to a further aspect the invention provides a method of chronotherapeutically treating a human or animal subject comprising the step of orally administering a substance delivery device as defined in any of the above mentioned aspects.

According to a further aspect the invention provides a method of chronotherapeutically treating a human or animal subject comprising the step of rectally administering a substance delivery device as defined in any of the above mentioned aspects.

According to a further aspect the invention provides a method of chronotherapeutically treating a human or animal subject comprising the step of implanting a substance delivery device as defined in any of the above mentioned aspects.

The step of implanting the substance delivery device may comprise the step of subcutaneously injecting the device.

According to a further aspect the invention provides a pharmaceutical product comprising at least two substance delivery devices, at least one of which is defined in any of the above aspects.

Because of the small size of the substance delivery devices of the present invention, it is relatively convenient to administer a number of devices together or within a short interval. The devices may be made to release the beneficial substance at different times, in order to maximise the therapeutic effect to the subject.

The invention will now be described, by way of example only, with reference to the following diagrams:

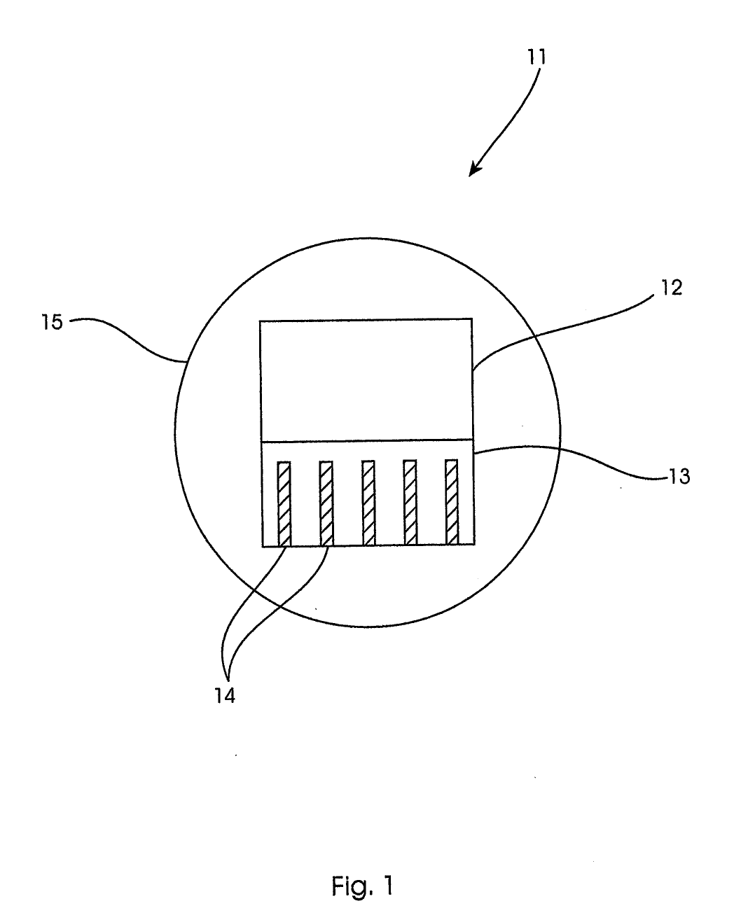

FIG. 1 is a schematic diagram of a substance delivery device according to the present invention;

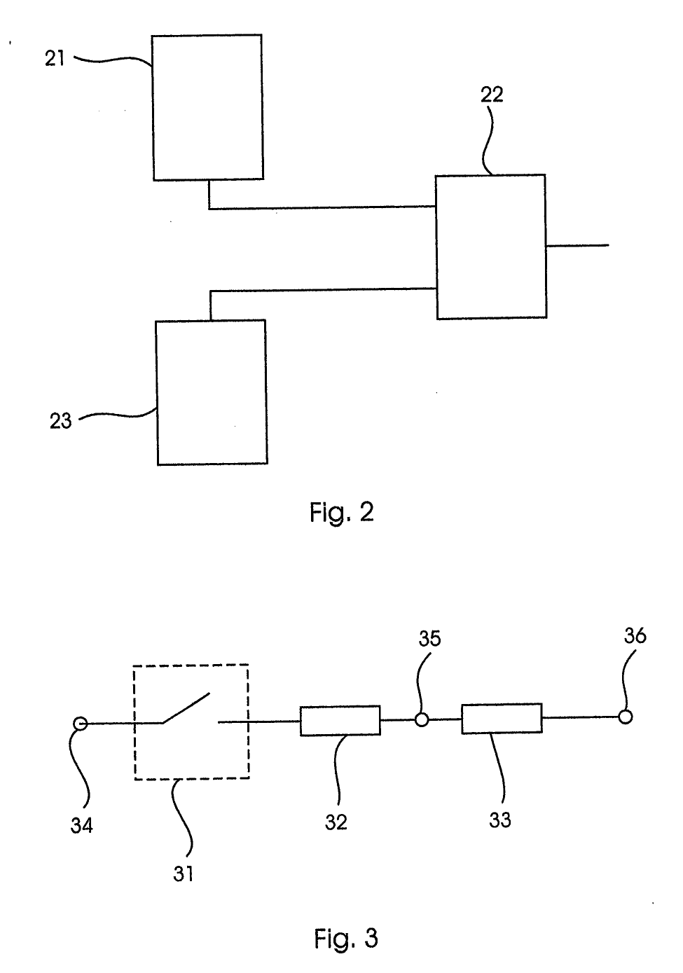

FIG. 2 is a schematic diagram of part of the electronic component that may form part of the substance delivery device shown in FIG. 1;

FIG. 3 is a schematic diagram of part of a device activation means that may form part of the FIG. 2 electronic component;

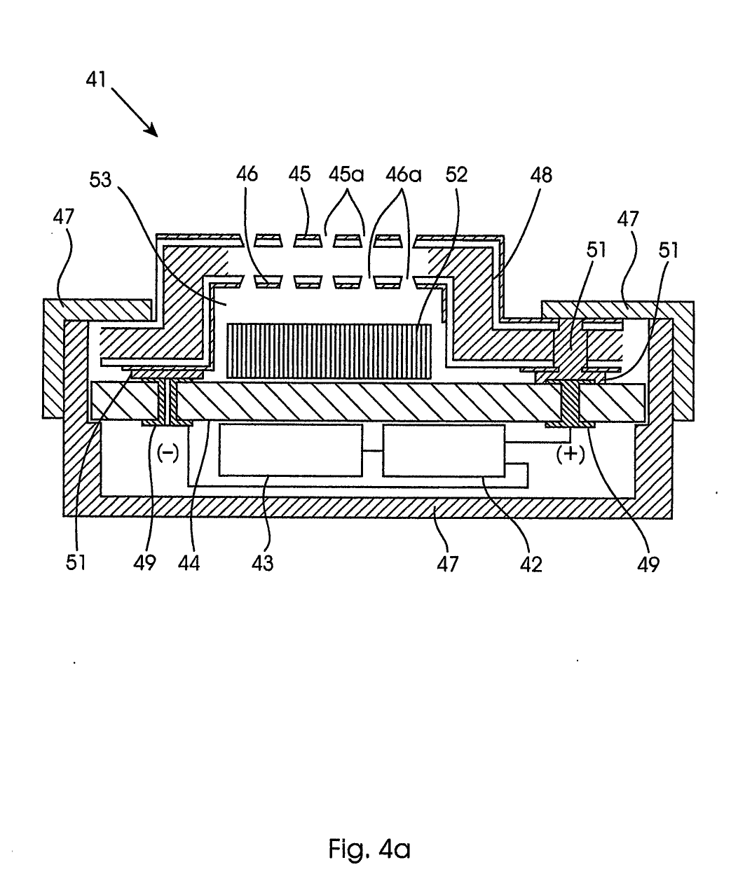

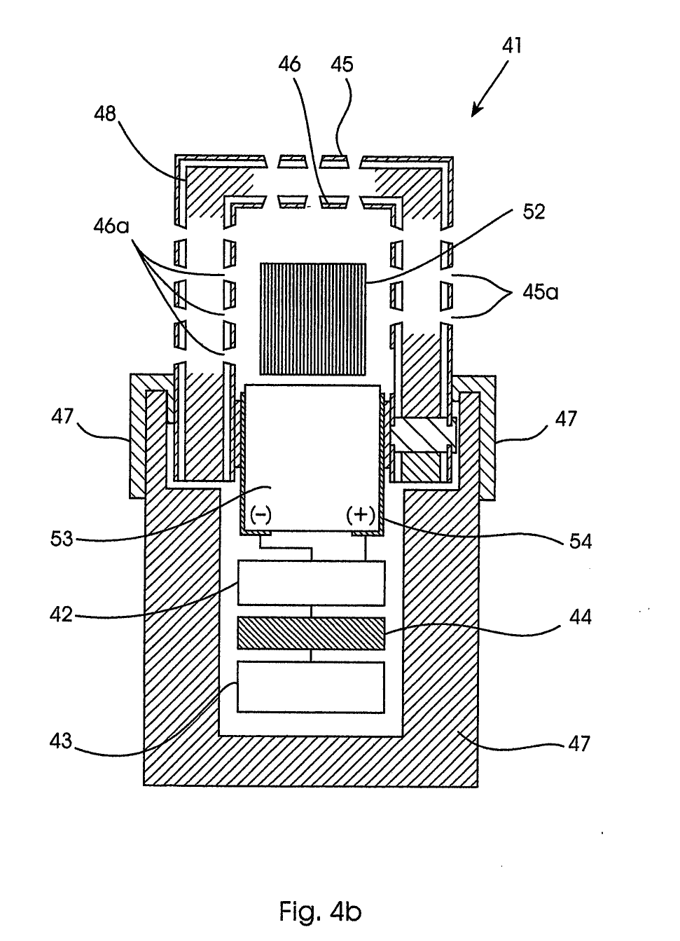

FIG. 4a is schematic diagram of a substance delivery device according to the present invention;

FIG. 4b is schematic diagram of a further substance delivery device according to the present invention;

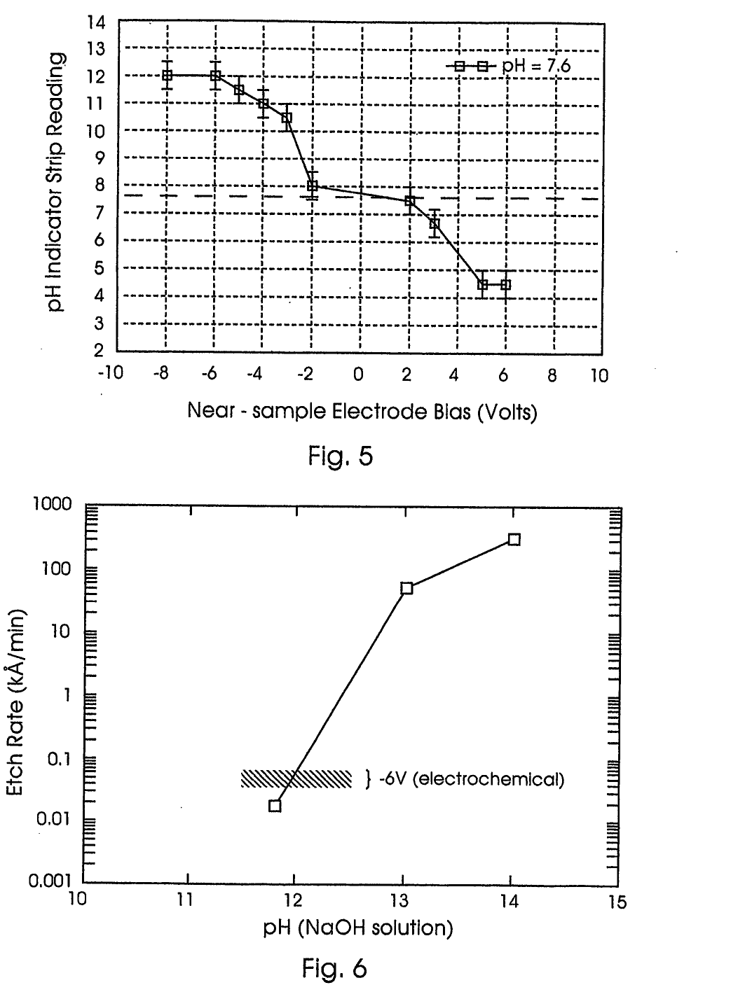

FIG. 5 contains a graph showing variation of pH with the potential of an electrode in the region of a porous silicon carrier component;

FIG. 6 contains a graph showing variation of etch rate, for a porous silicon carrier component, with pH

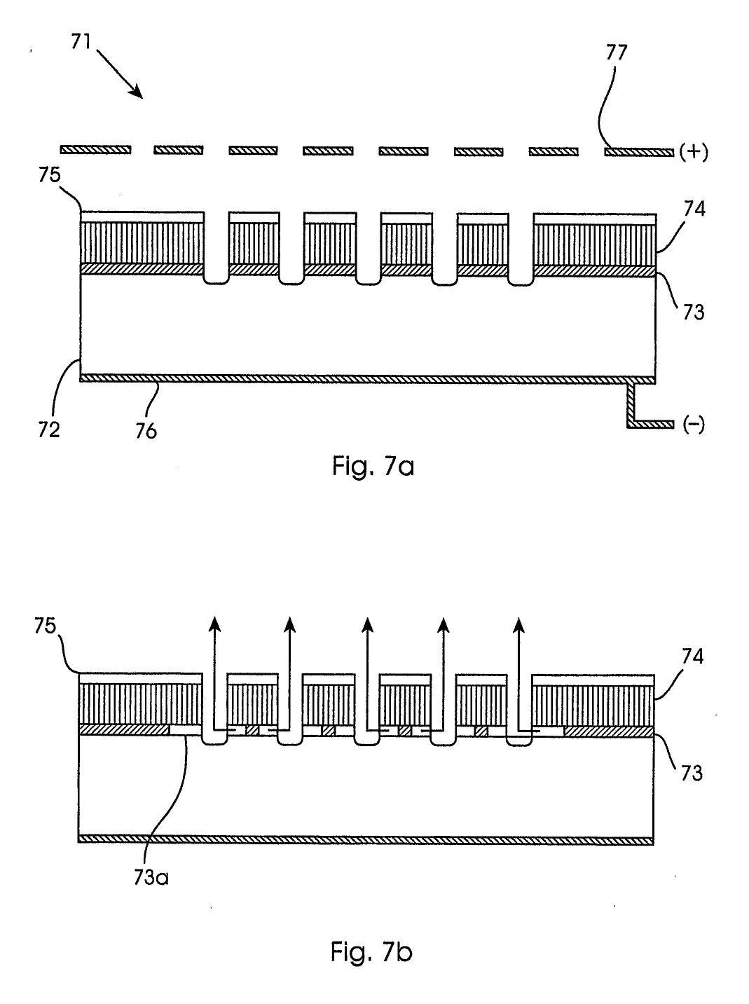

FIG. 7a is a schematic diagram of part of a substance delivery device, according to the present invention, prior to release of a beneficial substance from a porous silicon carrier material; and

FIG. 7b shows part of the FIG. 7a substance delivery device, according to the present invention, after release of the beneficial substance has begun to occur.

FIG. 1 shows a schematic diagram of a substance delivery device, generally indicated by 11, according to the present invention. The device comprises an electronic component 12, a carrier component 13, a beneficial substance 14, and an excipient coating 15. The carrier component comprises porous silicon and the beneficial substance 14 is located in the pores of the porous silicon. The substance delivery device 11 is suitable for oral consumption and the excipient coating 15 allows the device 11 to be relatively palatable to the subject.

FIG. 2 shows part of an electronic component 12 which comprises a substance delivery device activation means 21, beneficial substance release means 22, and a timer means 23. The electronic component also comprises a power supply, which is not shown in the figures, and may form part of the FIG. 1 substance delivery device.

FIG. 3 shows a schematic diagram of part of a device activation means. The FIG. 3 device activation means comprises a switch 31, a first resistor 32, a second resistor 33, a first switch electrode 34, a second switch electrode 35, and a third switch electrode 36. The FIG. 3 device activation means may form part of the FIG. 2 electronic component.

The timer means comprises a microcontroller chip and an oscillator or a resonator. For example the microcontroller may be a DS2417 Dallas RTC chip, and the oscillator may be an Epson 32.768 kHz quartz oscillator.

The microcontroller can be programmed so that the timer means generates a carrier time dependent electrical signal at a particular time, times, or range of times. For example the microcontroller may be programmed so that the timer means generates the electrical signal at 3 am.

The substance delivery device 11 may be fabricated in a factory, placed in a package, and it may be several weeks or months before the substance delivery device is consumed by the subject. During this interval the timer means generates the electrical signal, for example at 3 am every morning, even though it is still located in the packet. The generation ,of the electrical signal by the timer means has no effect on the carrier component or beneficial substance because the device has not been activated by the device activation means.

When the subject removes a substance delivery device, which comprises the FIG. 3 device activation means, and which does not comprise an excipient coating, from the packet and places it in their mouth, the subject's saliva causes the switch 31 to be closed. Electrical current passes through the saliva and between the first and third switch electrodes 34, 36. This causes the potential at the second switch electrode 35 to change.

The carrier activation means 22 detects the change of potential at the second switch electrode 35. If the potential at the second switch electrode 35 has changed, then and only then does the beneficial substance release means cause the release of the beneficial substance in response to the electrical signal from the timer means at the predetermined time of day.

The beneficial substance release means may comprise a means for heating the porous silicon. This means for heating the porous silicon is not shown in the figures. Porous silicon is resorbable in gastrointestinal environments. The rate of erosion may be increased by electrically heating the porous silicon in response to the carrier activation electrical signal. As the porous silicon erodes, in response to the heating initiated by the electrical signal from the timer means, so the beneficial substance 14 is released into the gastrointestinal environment.

The beneficial substance release means may comprise a means for applying a positive potential, relative to earth, to the porous silicon.

The erosion of porous silicon in the gastrointestinal environment may be accelerated by the application of a positive bias to the porous silicon. Therefore the application of such a bias, at a time dictated by the timer means, may cause porous silicon to erode, releasing the beneficial substance 14.

The substance delivery device 11 may further comprise NaHCO₃ located in the pores of the porous silicon. The application of a potential, relative to earth, to the porous silicon by the carrier activation means may cause the NaHCO₃ to be partly converted to gaseous C0₂. The consequent gas pressure may cause the beneficial substance 14, also located in the pores of the porous silicon, to be expelled from the carrier component 13, or it may cause the carrier component 13 to be fractured thereby releasing the beneficial substance 14.

FIG. 4a is a schematic diagram of a substance delivery device, generally indicated by 41, according to the present invention. The delivery device 41 comprises integrated circuitry 42, a lithium battery 43, a printed circuit board 44, an upper pH electrode 45, a lower pH electrode 46, and outer casing 47. The device also comprises an insulating plastic layer 48 that separates and insulates the upper pH electrode 45 from the lower pH electrode 46, together with electrical connectors 49 and conductive adhesive 51 that connect the integrated circuitry to the upper and lower pH electrodes 45, 46. A sample of porous silicon 52, from which the device is partly formed, is located in a cavity 53 formed between the lower pH electrode 46 and the printed circuit board 44. A beneficial substance, not shown in FIG. 4a, may be located in the pores of the porous silicon 52.

The pH electrodes 45, 46, which may be formed by standard techniques, may comprise platinum, silver, or gold and are perforated to form electrode holes 45a and 46a.

The integrated circuitry 42 comprises a microcontroller, RTC module, and a current/voltage driver for the electrochemical cell electrodes, none of the individual components of the integrated circuitry being shown in FIG. 4a. The integrated circuitry 42 and battery 43 are mounted onto one side of the circuit board 44. The printed circuit board 44 may be partly formed from fibreglass, ceramic, or plastic, and have metal tracks, not shown, for electrically connecting the components of the integrated circuitry 42, the components of the integrated circuitry 42 being wire bonded or bump bonded to the circuit board 44 by standard techniques.

The upper and lower pH electrodes 45, 46 and insulating layer are mounted on the circuit board 44 using conductive adhesive. The outer casing 47 is then put in place to protect the battery 42 and integrated circuitry 42 from the biological environment into which the delivery device may be introduced. The outer casing 47 may be formed from an electrically insulating material. However, for some embodiments, the integrated circuitry may have to be connected to a reference electrode, not shown in the FIG. 4a, and this reference electrode may be mounted on the outer casing.

FIG. 4b is a schematic diagram of a substance delivery device according to the present invention. The device and its components are very similar to those shown in FIG. 4a, therefore the same numbering system has been adopted. In the FIG. 4b device, the lithium battery 43 is mounted on the opposite side of the circuit board 44 to the integrated circuitry 42. This allows the FIG. 4b delivery device to have a smaller diameter, and increased length, relative to that of FIG. 4a. Additional perforations 45a, 46a are formed in the cylindrical walls of the FIG. 4b electrodes 45 and 46. Electrical connection between the integrated circuitry 42 and the pH electrodes 46 are made via a plug 53 having a metalised surface 54. The connection between the integrated circuitry 42 and the metalised surface 54 can be made using spring loaded contacts, which are not shown in FIG. 4b.

A beneficial substance may be located in the pores of the porous silicon carrier material from which the porous silicon sample 52 is formed. The beneficial substance is not shown in FIG. 4.

When immersed in the gastrointestinal environment, each of the delivery devices 41 will form an electrochemical cell. The battery 43 and integrated circuitry 42 are constructed such that the upper pH electrode 45 is positively biased, and the lower pH electrode 46 is negatively biased. This biasing results in the pH in the region of the porous silicon sample, which is occupied by gastrointestinal fluid, being higher relative to gastrointestinal fluid outside the delivery device 41. The erosion rate of the porous silicon increases as the pH of the environment decreases, thereby increasing the rate of release of a drug contained in its pores. The drug is able to enter the GI tract of the subject through the openings 45a and 46a in the pH electrodes 45, 46.

FIG. 5 shows the pH, in the region of a sample of porous silicon, as a function of the potential of a platinum mesh electrode placed in close proximity to a sample of porous silicon. The platinum electrode was approximately 2 mm from the surface of the porous silicon sample.

The FIG. 5 experiment was performed using tap water having a pH of 7.6, when no potential was applied. The experiment shows that as the electrode potential decreases, so the pH in the region of the porous silicon increases.

In a separate experiment, the effect of etch rate, for a sample of porous silicon, was measured as a function of pH. The results are presented in FIG. 6, and show that etch rate increases with pH. The variation of pH for the FIG. 6 results was obtained by altering the concentration of NaOH in a NaOH buffer solution.

FIG. 7a is a schematic diagram of part of a substance delivery device, according to the present invention, generally indicated by 71. The delivery device 71 comprises a non-porous substrate 72, a high porosity porous silicon region 73, a low porosity porous silicon region 74, a capping layer 75, a negative pH electrode 76, a positive pH electrode 77. The non-porous substrate 72 may comprise n+ or p+ bulk crystalline silicon. A beneficial substance, not shown in FIG. 7, is initially located in the pores of the low porosity region 74. When a potential difference is applied to the positive and negative electrodes 76, 77 the pH in the region of the high porosity region increases, causing increased dissolution relative to the low porosity region. The beneficial substance, located in the low porosity region 74, is initially prevented from escape by the capping layer 75, and high porosity porous silicon 73. The capping layer may comprise silicon oxide, the low porosity region 74 may comprise porous silicon having a porosity between 20% and 60%, the high porosity region 73 may have a porosity between 40% and 80%. FIG. 7b shows part of the FIG. 7a substance delivery device after the high porosity region 73 has been eroded. The increased dissolution of the high porosity region 73 may allow release of the beneficial substance through the resulting channel 73a, shown in FIG. 7, between the substrate 72 and the low porosity region 74.

1. A substance delivery device comprising: (a) an electronic timer means for generating a time dependent electronic signal, (b) a substance delivery device activation means for activating the substance delivery device, (c) a beneficial substance, and (d) a beneficial substance release means for releasing the beneficial substance in response to the time dependent electronic signal, once the substance delivery device has been activated.

2. A substance delivery device according to claim 1 characterised in that timer means, when in use, has a construction and/or has been programmed to generate an electronic signal having a periodic variation with time.

3. A substance delivery device according to claim 2 characterised in that the period of variation is selected from one or more of: 12 hours, 1 day, 2 days, 1 week, 2 weeks, and 1 month.

4. A substance delivery device according to claim 1 characterised in that the device activation means comprises an activation electrical switch having an on state and an off state, the substance delivery device having a construction such that, when in use, the beneficial substance release means releases the beneficial substance once the electrical switch is in an on state.

5. A substance delivery device according to claim 4 characterised in that the substance delivery device, and electrical activation means each have a construction such that, when the substance delivery device has been immersed in a physiological electrolyte, the activation electrical switch is switched to an on state by contact between the electrical activation means and the physiological electrolyte.

6. A substance delivery device according to claim 1 characterised in that the beneficial substance release means comprises porous silicon, the beneficial substance being located in at least some of the pores of the porous silicon.

7. A substance delivery device according to claim 6 characterised in that the beneficial substance release means further comprises two or more pH electrodes, and a means for applying a potential difference between the two, or at least two of, electrodes, the arrangement of the pH electrodes and potential difference being such that, when immersed in a physiological electrolyte, OH— ions are attracted towards at least part of the porous silicon by the electrical field generated by the pH electrodes.

8. A substance delivery device according to claim 7 characterised in that the substance delivery device has a construction such that none of the pH electrodes is in direct physical contact with porous silicon.

9. A packaged substance delivery device comprising a package in which at least one substance delivery device, as claimed in claim 1, is disposed.

10. A packaged substance delivery device according to claim 9, characterised in that the or at least one of the device activation has a construction such that removal of the device from the package activates the delivery device.
